(19) United States
(12) Patent Application Publication
(10) Pub. No.: US 2025/0252567 A1
(43) Pub. Date: Aug. 7, 2025

(54) SYSTEMS AND METHODS FOR IDENTIFYING AND CORRECTING DEFORMITIES OF THE LOWER EXTREMITIES

(71) Applicant: Paragon 28, Inc., Englewood, CO (US)

(72) Inventor: Andrew Julian GOLDBERG, Radlett (GB)

(73) Assignee: Paragon 28, Inc., Englewood, CO (US)

(21) Appl. No.: 19/044,145

(22) Filed: Feb. 3, 2025

Related U.S. Application Data
(60) Provisional application No. 63/548,961, filed on Feb. 2, 2024.

(51) Int. Cl. G06T 7/00; G16H 20/40; G16H 30/40

(52) U.S. Cl. CPC G06T 7/0012; G16H 20/40; G16H 30/40; G06T 2207/30008

(57) ABSTRACT

A process for identifying a deformity includes the steps of receiving patient input data showing patient anatomy, segmenting the patient input data, and generating a three-dimensional model of the patient anatomy. The process also includes identifying one or more points on the patient anatomy, collecting one or more measurements incorporating the one or more points on the patient anatomy, determining, based on the one or more measurements, if a deformity is present in the patient anatomy, and recommending one or more actions to correct the deformity of the patient anatomy.

### CROSS REFERENCE TO RELATED APPLICATION

This application perfects U.S. Provisional Patent Application No. 63/548,961, filed on Feb. 2, 2024, and entitled “Systems and Methods For Identifying and Correcting Deformities of the Lower Extremities”, the entirety of which is hereby incorporated herein by reference.

### FIELD OF THE DISCLOSURE

The present disclosure relates systems and methods to be implemented in planning and performing surgical procedures. The present disclosure relates to podiatric and orthopedic systems and methods to be implemented in various procedures of the foot, ankle, or other anatomy. More specifically, but not exclusively, the present disclosure relates to systems and methods to be implemented in planning and performing osteotomy procedures of the foot and/or ankle.

### BACKGROUND

Many currently available surgical systems and methods do not completely address the needs of patients. Additionally, many currently available surgical systems and methods fail to account for properties of foot and ankle anatomy and accordingly can decrease favorability of the outcome for the patient.

### SUMMARY

The present disclosure is directed toward implants, instruments, and surgical methods to be implemented in procedures of the foot and ankle.

A first aspect of the present disclosure is directed to a process for identifying a deformity. The process includes receiving patient input data showing patient anatomy, segmenting the patient input data, collecting one or more measurements from the three-dimensional model of the patient anatomy, and determining if a deformity is present in the patient anatomy.

According to the first aspect of the present disclosure, the patient anatomy includes a tibia of a patient.

According to the first aspect of the present disclosure, the process includes identifying a proximal portion of the tibia in the three-dimensional model, and identifying a distal portion of the tibia in the three-dimensional model.

According to the first aspect of the present disclosure, the process includes identifying one or more anatomic or geometric planes relative to the tibia in the three-dimensional model.

According to the first aspect of the present disclosure, the process includes identifying one or more points on the proximal portion of the tibia in the three-dimensional model, and identifying one or more points on the distal portion of the tibia in the three-dimensional model.

According to the first aspect of the present disclosure, the process includes determining the position of the one or more points on the proximal portion of the tibia relative to the one or more anatomic landmarks or geometric planes in the three-dimensional model, and determining the position of the one or more points on the distal portion of the tibia relative to the one or more anatomic landmarks or geometric planes in the three-dimensional model.

According to the first aspect of the present disclosure, the process includes calculating a difference between the determined position of the one or more points on the proximal portion of the tibia relative to the one or more anatomic landmarks or geometric planes in the three-dimensional model and a normal position of the one or more points on the proximal portion of the tibia relative to the one or more anatomic landmarks or geometric planes.

According to the first aspect of the present disclosure, the process includes calculating a difference between the determined position of the one or more points on the distal portion of the tibia relative to the one or more anatomic landmarks or geometric planes in the three-dimensional model and a normal position of the one or more points on the distal portion of the tibia relative to the one or more anatomic landmarks or geometric planes.

According to the first aspect of the present disclosure, the process includes determining, based on the calculated difference between the determined position of the one or more points on the proximal portion of the tibia relative to the one or more anatomic landmarks or geometric planes in the three-dimensional model and the normal position of the one or more points on the proximal portion of the tibia relative to the one or more anatomic landmarks or geometric planes, if a deformity is present in the proximal portion of the tibia.

According to the first aspect of the present disclosure, the process includes determining, based on the calculated difference between the determined position of the one or more points on the distal portion of the tibia relative to the one or more anatomic landmarks or geometric planes in the three-dimensional model and the normal position of the one or more points on the distal portion of the tibia relative to the one or more anatomic landmarks or geometric planes, if a deformity is present in the proximal portion of the tibia.

According to the first aspect of the present disclosure, the process includes classifying a deformity identified in the proximal portion of the tibia.

According to the first aspect of the present disclosure, the process includes classifying a deformity identified in the distal portion of the tibia.

According to the first aspect of the present disclosure, the process includes recommending, based on the deformity identified in the distal portion of the tibia, one or more corrective procedures to be performed to the tibia.

A second aspect of the present disclosure is directed to a process for determining an optimal osteotomy angle. The process includes receiving patient input data showing patient anatomy, segmenting the patient input data, generating a three-dimensional model of the patient anatomy, identifying one or more anatomic structures in the three-dimensional model, identifying one or more anatomic landmarks on the one or more anatomic structures in the three-dimensional model, determining a desired correction to be achieved from an osteotomy, and determining an optimal osteotomy angle.

According to the second aspect of the present disclosure, the desired correction includes a desired rotational correction of a distal portion of a tibia relative to a proximal portion of the tibia.

According to the second aspect of the present disclosure, the process includes determining an optimal osteotomy point, wherein the optimal osteotomy point is positioned along the long axis of the tibia.

According to the second aspect of the present disclosure, the process includes determining, based on the osteotomy point, total surface area of overhang and underhang resulting from rotation of the distal portion of the tibia relative to the proximal portion of the tibia for an osteotomy performed at a first osteotomy angle across the optimal osteotomy point.

According to the second aspect of the present disclosure, the process includes determining, based on the osteotomy point, total surface area of overhang and underhang resulting from rotation of the distal portion of the tibia relative to the proximal portion of the tibia for an osteotomy performed at a second osteotomy angle across the optimal osteotomy point.

According to the second aspect of the present disclosure, the process includes determining, based on the total surface area of overhang and underhang for osteotomies performed at the first and second osteotomy angles, an optimal osteotomy angle, wherein the optimal osteotomy angle is the angle of the first and second osteotomy angles with the lease total surface area of overhang and underhang.

A third aspect of the present disclosure is directed to a process for identifying a deformity. The process includes receiving patient input data showing patient anatomy, segmenting the patient input data, generating a three-dimensional model of the patient anatomy, identifying one or more points on the patient anatomy, collecting one or more measurements incorporating the one or more points on the patient anatomy, determining, based on the one or more measurements, if a deformity is present in the patient anatomy, and recommending one or more actions to correct the deformity of the patient anatomy.

### BRIEF DESCRIPTION OF THE DRAWINGS

The accompanying drawings, which are incorporated in and constitute a part of the specification, illustrate embodiments of the inventions and together with the detailed description herein, serve to explain the principles of the inventions. It is emphasized that, in accordance with the standard practice in the industry, various features may or may not be drawn to scale. In fact, the dimensions of the various features may be arbitrarily increased or reduced for clarity of discussion. The drawings are only for purposes of illustrating embodiments of inventions of the disclosure and are not to be construed as limiting the inventions.

FIG. 1 is a flowchart showing an exemplary process, in accordance with the present disclosure;

FIG. 2 is a flowchart showing an exemplary process, in accordance with the present disclosure;

FIG. 3 is schematic diagram of various exemplary planes and landmarks of a lower leg of a patient, in accordance with the present disclosure;

FIG. 4 is a schematic diagram of exemplary landmarks and an exemplary osteotomy plane of the lower leg of the patient, in accordance with the present disclosure;

FIG. 5 is a schematic diagram of exemplary landmarks and an exemplary osteotomy plane of the lower leg of the patient, in accordance with the present disclosure;

FIG. 6 is a schematic diagram of exemplary landmarks and an exemplary osteotomy plane of the lower leg of the patient, in accordance with the present disclosure;

FIG. 7 is a schematic diagram of exemplary landmarks and an exemplary osteotomy plane of the lower leg of the patient, in accordance with the present disclosure;

FIG. 8 is a schematic diagram of exemplary landmarks and an exemplary osteotomy plane of the lower leg of the patient, in accordance with the present disclosure;

FIG. 9 is an exemplary cross-section of a tibia of the patient relative to the exemplary osteotomy of FIG. 4, in accordance with the present disclosure;

FIG. 10 is an exemplary cross-section of the tibia of the patient relative to the exemplary osteotomy of FIG. 5, in accordance with the present disclosure;

FIG. 11 is an exemplary cross-section of the tibia of the patient relative to the exemplary osteotomy of FIG. 6, in accordance with the present disclosure;

FIG. 12 is an exemplary cross-section of the tibia of the patient relative to the exemplary osteotomy of FIG. 7, in accordance with the present disclosure;

FIG. 13 is an exemplary cross-section of the tibia of the patient relative to the exemplary osteotomy of FIG. 8, in accordance with the present disclosure; and

FIG. 14 is an example computer system to incorporate and/or use aspects described herein; in accordance with the present invention.

### DETAILED DESCRIPTION

In this detailed description and the following claims, the words proximal, distal, anterior or plantar, posterior or dorsal, medial, lateral, superior and inferior are defined by their standard usage for indicating a particular part or portion of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, specifically in regards to the foot, the term “dorsal” refers to the top of the foot and the term “plantar” refers to the bottom of the foot.

Similarly, positions or directions may be used herein with reference to anatomical structures or surfaces. For example, as the current implants, devices, instrumentation, and methods are described herein with reference to use with the bones of the foot, the bones of the foot, ankle and lower leg may be used to describe the surfaces, positions, directions or orientations of the implants, devices, instrumentation and methods. Further, the implants, devices, instrumentation, and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, instrumentation, and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, instrumentation, and methods, and the aspects, components, features and the like thereof, described herein with respect to the right foot may be mirrored so that they likewise function with the left foot. Further, the implants, devices, instrumentation, and methods, and the aspects, components, features and the like thereof, disclosed herein are described with respect to the foot for brevity purposes, but it should be understood that the implants, devices, instrumentation, and methods may be used with other bones of the body having similar structures.

Osteotomies are commonly performed surgical procedures that include performing at least one cut to bony anatomy, typically in order to address one or more deformities. Procedures involving osteotomies are often performed in order to correct coronal (e.g., frontal) or sagittal (e.g., lateral) plane deformities in bony anatomy of a patient. Osteotomies are often performed to the tibia and/or femur of patients in order to correct rotational deformities of these bones, for example when a distal portion of the bone has rotated (e.g., a torsional deformity in the tibia or a versional deformity in the femur), typically laterally, relative to a proximal portion of the bone. In these corrective procedures, an osteotomy cut is performed at some point along the length of the bone, and the distal segment of the bone (as well as all anatomy distal of the osteotomy plane) is rotated to correct the rotational deformity, and the two segments of the bone are then fixed (using a plate or other fixation hardware) to maintain the correction. Osteotomies are commonly performed at various oblique angles relative to the long axes of these bones and, as such, various protrusions of the bone can either be created or repositioned during this procedure. For example, if an osteotomy plane bisects an osteophyte protruding from a lateral portion of the tibia, rotation of the distal portion of the tibia to correct the deformity may reposition a distal portion of the osteophyte and as such may interfere with the fibula and/or adjacent soft tissue structures. Accordingly, it is desirable to optimize osteotomy sites and angles so as to minimize any interference with adjacent anatomy caused by changing or repositioning the outer perimeter (and features thereof) of the distal segment of the bone.

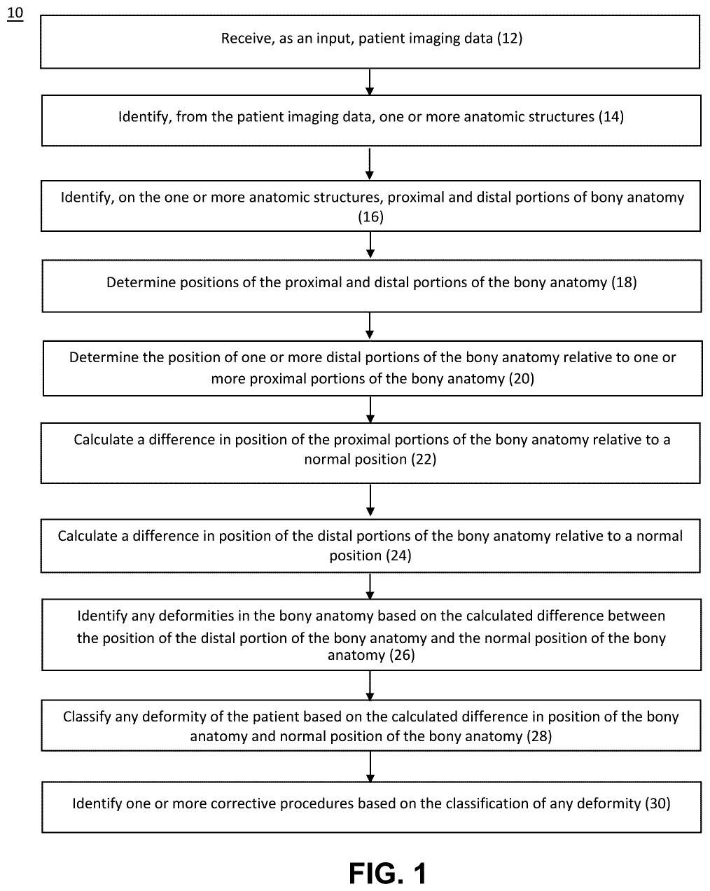

Referring now to FIG. 1, a process 10 of identifying and classifying a tibial deformity (referred to hereinafter as “process 10”) is shown, according to an exemplary embodiment. The process 10 may be performed entirely or in part using one or more software systems or programs, for example with one or more inputs provided to the software system and one or more outputs generated by the software system. Further, it should be understood that one or more of the steps of the process 10 may be repeated, omitted, or performed in an alternate order to that shown in the exemplary embodiment of FIG. 1.

The process 10 is shown to include a step 12 of receiving, as an input, patient imaging data, according to an exemplary embodiment. The patient imaging data may include one or more x-rays/radiographic images, CT data (including weight-bearing CT (WBCT) data), MRI data, or other medical imaging data. The patient imaging data may also include various anatomy of the patient, for example one or more extremities of the patient such as a leg. In some aspects, the patient imaging data may show anatomy extending from a central portion of the patient (e.g., the abdomen, pelvis, etc.) to a distal-most point of an extremity (e.g., distal phalanxes of the foot), or may show anatomy of a central portion of an extremity (e.g., from midfoot to knee). Such data may be uploaded by a physician via a web portal (e.g., with cloud access), or may otherwise be input into the system or program.

The process 10 is shown to include a step 14 of identifying, from the patient imaging data, one or more anatomic structures, according to an exemplary embodiment. In some aspects, the step 14 may be performed at least in part by a software program, for example an automatic segmentation tool. For example, after the patient imaging data is uploaded, a software program may segment the imaging data to identify one or more anatomic structures shown in the patient imaging data. In some aspects, this software program may generate a three-dimensional model of the anatomy shown in the patient imaging data, with the model being manipulatable by a physician and/or personnel involved in planning one or more procedures involving the anatomy. For example, a physician may simulate one or more cuts to the anatomy, or may collect various data points (e.g., measurements of one point of the anatomy relative to another point of the anatomy or an identified axis or plane). Step 14 may include identifying various bony structures of the foot/ankle, the tibia, fibula, patella, femur, pelvis, as well as soft tissue structures adjacent to the aforementioned bony anatomy.

The process 10 is shown to include a step 16 of identifying, on the one or more anatomic structures, proximal and distal portions of bony anatomy, according to an exemplary embodiment. The step 16 may include identifying a proximal section of the tibia of the patient and a distal section of the tibia of the patient. In some aspects, this may be performed by identifying one or more prominent features of the respective tibial portions, for example the medial malleolus of the distal portion and various osteophytes or condyles of the proximal portion. The step 16 may also include identifying one or more axes of the anatomy or common anatomical planes, for example the long axis (e.g., longitudinal axis) of the tibia, as well as a ground plane, sagittal plane, coronal plane, and/or other similar axes/planes.

The process 10 is shown to include a step 18 of determining the positions of the proximal and distal portions of the bony anatomy, according to an exemplary embodiment. Step 10 may include identifying a point on the upper and lower portions of the tibia, for example the medial malleolus, an osteophyte, a point on the outer surface of the bone, or other point, and collecting one or more measurements of the points relative to one or more planes or axes (e.g., the planes and axes mentioned previously). For example, a point on the proximal portion of the tibia may be measured to be 5 degrees lateral relative to the parasagittal plane bisecting the tibia in anterior-posterior directions. Similarly, a point on the distal portion of the tibia may be similarly measured rotationally relative to the parasagittal plane, which may be rotated 20 degrees lateral relative to the parasagittal plane.

The process 10 is shown to include a step 20 of determining the position of one or more distal portions of the bony anatomy relative to one or more proximal portions of the bony anatomy, according to an exemplary embodiment. The measured positions of the one or more points on the upper and lower portions of the tibia may be analyzed relative to normal anatomy. For example, the point on the proximal portion of the tibia measured to be 5 degrees lateral relative to the parasagittal plane may fall within a normal/accepted range (e.g. 0-10 degrees) for that point relative to normal anatomy (e.g., anatomy in which a deformity is not present). However, the point on the distal portion of the tibia measured to be 20 degrees lateral relative to the parasagittal plane may fall outside of a normal/accepted range (e.g., 5-15 degrees) for that point relative to normal anatomy. In some aspects, the software system/program may include artificial intelligence (AI) and/or machine learning (ML) aspects/models which have been trained to distinguish abnormal anatomy of the tibia from normal anatomy of the tibia, for instance model(s) that have been trained on training data such as measurements and other data relating to anatomical position, shape, dimensions, and/or other features. The training could be supervised, e.g., based on training data labeled as abnormal, normal, and/or other classifications, or unsupervised to distinguish between abnormal and normal (or other classifications of) anatomy. In some aspects, the software program/system may collect the measurements using the generated 3D model based on the patient imaging data.

The process 10 is shown to include a step 22 of calculating a difference in position of the proximal portions of the anatomy relative to a normal position, according to an exemplary embodiment. With continuing reference to the example provided with reference to steps 18 and 20, the point on the proximal portion of the tibia (5 degrees laterally-rotated from the parasagittal plane) may be calculated to be equidistant from the upper and lower limits (0-10 degrees) and thus within the accepted/normal range for distal tibial anatomy, and the calculation may be performed by the software program/system.

The process 10 is shown to include a step 24 of calculating a difference in position of the distal portions of the bony anatomy relative to a normal position, according to an exemplary embodiment. With continuing reference to the example provided with reference to steps 18, 20, and 22, the point on the distal portion of the tibia may be calculated to be laterally rotated, relative to the parasagittal plane, 5 degrees outside of the accepted/normal range (5-15 degrees) for distal tibial anatomy. In some aspects, this calculation may be performed by the software program/system.

The process 10 is shown to include a step 26 of identifying any deformities in the bony anatomy based on the calculated difference between the position of the distal portion of the bony anatomy and the normal position of the bony anatomy, according to an exemplary embodiment. With continued reference to the previous example of step 24, step 26 may include identifying the distal portion of the tibia of the patient as having a deformity relative to a normal tibia based on the deviation of the measurement of the point on the distal portion of the tibia from the accepted range. Should such a determination of a deformity be made (which may be performed by the software program/system), the physician may be notified that a deformity is present.

The process 10 is shown to include a step 28 of classifying any deformity of the patient based on the calculated difference in position of the bony anatomy and normal position of the bony anatomy, according to an exemplary embodiment. Step 28 may include the software program/system classifying the deformity determined in step 26 based on how far from the accepted/normal ranged the measurement of the point on the distal tibia deviates. For example, the deviation of 0-10 degrees from the accepted range may be classified as a “moderate deformity”, whereas a deviation of greater than 10 degrees from the accepted/normal range may be classified as a “severe deformity.”

The process 10 is shown to include a step 30 of identifying one or more corrective procedures based on the classification of any deformity, according to an exemplary embodiment. Based on the classification of the deformity in step 28, the software program/system may recommend one or more corrective actions/procedures that may be considered by the physician to correct the tibial deformity of the patient. For example, a recommendation of a supramalleolar osteotomy with 5 degrees of medial rotational correction may be recommended based on the 5 degrees of deviation outside of the normal/accepted range for the measurement of the point on the distal tibia (relative to the parasagittal plane). In some aspects, different procedures may be recommended for a severe deformity than for a moderate deformity.

In some aspects, patients may present with symptoms similar to those of a tibial rotational (torsional) deformity, but a rotational deformity may not be present in the tibia and may not be determined to be present based on measurements collected and analyzed in the process 10. Similarly, patients may present with symptoms of other conditions, for example flatfoot/progressive collapsing foot deformity (PCFD), but have not exhibited any anatomical deformities consistent with flatfoot/PCFD. Rather, such a patient may have an anatomical deformity of the tibia (e.g., a rotational deformity) which may be identified in the process 10 so as to prevent the patient from undergoing a procedure to address a flatfoot/PCFD deformity which may not actually be present. Further, in some aspects the patient may present with symptoms similar to those of flatfoot/PCFD and/or a rotational (torsional) tibial deformity, but based on the analysis of the process 10, not have any deformity in the corresponding anatomy. In such an instance, the process 10 may include an analysis of a femur and/or pelvis of the patient, where the process 10 is repeated for the femur of the patient (e.g., to identify any rotational/torsional deformity of the distal portion of the femur relative to the proximal portion) just as for the tibia in the exemplary scenario described herein.

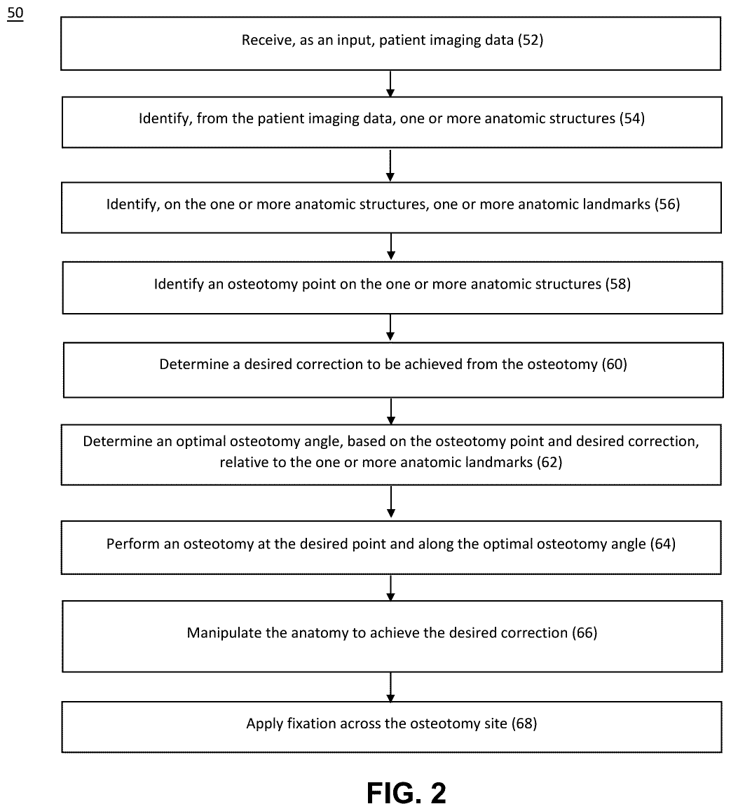

Referring now to FIG. 2, a process 50 of optimizing an osteotomy (referred to hereinafter as “process 50”) is shown, according to an exemplary embodiment. The process 50 may be performed entirely or in part using one or more software systems and/or programs, for example with one or more inputs provided to the software system and one or more outputs generated by the software system. Further, it should be understood that one or more of the steps of the process 50 may be repeated, omitted, or performed in an alternate order to that shown in the exemplary embodiment of FIG. 2.

The process 50 is shown to include a step 52 of receiving, as an input, patient imaging data, according to an exemplary embodiment. The patient imaging data may include one or more x-rays/radiographic images, CT data (including weight-bearing CT (WBCT) data), MRI data, or other medical imaging data. The patient imaging data may also include various anatomy of the patient, for example one or more extremities of the patient such as a leg. In some aspects, the patient imaging data may show anatomy extending from a central portion of the patient (e.g., the abdomen, pelvis, etc.) to a distal-most point of an extremity (e.g., distal phalanxes of the foot), or may show anatomy of a central portion of an extremity (e.g., from midfoot to knee). Such data may be uploaded by a physician via a web portal (e.g., with cloud access), or may otherwise be input into a system.

The process 50 is shown to include a step 54 of identifying, from the patient imaging data, one or more anatomic structures, according to an exemplary embodiment. In some aspects, step 54 may be performed at least in part by a software program, for example an automatic segmentation tool. For example, after the patient imaging data is uploaded, a software program may segment the imaging data to identify one or more anatomic structures shown in the patient imaging data. In some aspects, this software may generate a three-dimensional model of the anatomy shown in the patient imaging data, with the model being manipulatable by the physician and/or personnel involved in planning one or more procedures involving said anatomy. For example, the physician may simulate one or more cuts to the anatomy, or may collect various data points (e.g., measurements of one point of the anatomy relative to another point of the anatomy or an identified axis or plane). Step 54 may include identifying various bony structures of the foot/ankle, the tibia, fibula, patella, femur, pelvis, as well as soft tissue structures adjacent to the aforementioned bony anatomy.

The process 50 is shown to include a step 56 of identifying, on the one or more anatomic structures, one or more anatomic landmarks, according to an exemplary embodiment. Step 56 may also include identifying one or more axes of the anatomy or common anatomical planes, for example the long axis (e.g., longitudinal axis) of the tibia, as well as a ground plane, sagittal plane, coronal plane, and/or other similar axes/planes.

The process 50 is shown to include a step 58 of identifying an osteotomy point on the one or more anatomic structures, according to an exemplary embodiment. In some aspects, the osteotomy point may be provided as an input by the physician, for example 10 cm from the distal-most point of the tibia, where the point is placed along the axis of the tibia. However, in some aspects, the osteotomy point may be determined by the software program/system. For example, the software may be configured to identify one or more deformities of the tibia (e.g., for example as done in the process 10) and based on the one or more deformities, identify an optimal osteotomy point along the axis of the tibia. The optimal osteotomy point may be based on access to the tibia relative to adjacent anatomy (e.g., soft tissue, etc.), anatomic position of deformity, severity of deformity, and/or other similar factors.

The process 50 is shown to include a step 60 of determining a desired correction to be achieved from the osteotomy, according to an exemplary embodiment. The desired correction to be achieved from the osteotomy may be determined (either by the physician or the software program/system) according to measurement and/or classification of a deformity, for example rotation of a distal portion of the tibia relative to a proximal portion of the tibia (e.g., tibial torsion) as described with reference to the process 10. For example, if a deformity of 5 degrees of lateral rotation is measured (e.g., distal tibia rotated 5 degrees relative to normal position), the desired correction may be 5 degrees of medial rotation (e.g., such that the measured value falls within the normal/accepted range after the osteotomy has been performed). In some aspects, the desired correction may also consider other factors, for example a threshold of rotational correction at which a fibular osteotomy is required (where a physician may desire to perform or avoid performing a fibular osteotomy), patient demographic information, concurrent/subsequent procedures, and/or other similar information.

The process 50 is shown to include a step 62 of determining an optimal osteotomy angle, based on the osteotomy point and desired correction, relative to the one or more anatomic landmarks, according to an exemplary embodiment. The determination of the optimal osteotomy angle may be performed by the software program system (which may or may not receive input/parameters from the physician), which incorporates various measurements, calculations, and other factors described previously herein and determined in the processes 10 and 50. In some aspects, step 62 may include one or more simulations performed by the software program/system, for example to analyze the cross-sectional geometry of the tibia should an osteotomy be made across the osteotomy point in the longitudinal axis at various angles. For example, step 62 may include simulating osteotomies made at angles ranging from 0 degrees (e.g., perpendicular to the axis of the tibia) to 90 degrees (parallel with the axis of the tibia), with the resulting cross-sectional geometries of the tibia analyzed for each incremental angular osteotomy. Further, based on the desired rotational correction from the osteotomy, the software program/system may calculate the surface area and/or volume of any overhang or underhang (e.g., prominences resulting from rotation of the distal portion of the tibia (and anatomy distal of the osteotomy point) relative to the proximal portion of the tibia) resulting from a given osteotomy angle and rotational correction.

For example, an osteotomy performed at 0 degrees (e.g., perpendicular to the axis of the tibia) may produce a greater total surface area of overhang and underhang with 5 degrees of medial rotational correction than an osteotomy performed at 30 degrees relative to the axis of the tibia (and paired with the same medial rotational correction, where the 30 degree osteotomy angle provides the least overhang/underhang surface area of any osteotomy angle). Accordingly, the software program/system may recommend an optimal osteotomy angle of 30 degrees so as to prevent any soft tissue irritation/obstruction that may be caused by unnecessary overhang/underhang should the osteotomy be performed at an alternative osteotomy angle. In some aspects, the optimal osteotomy angle may also be determined with start and end points relative to the tibia, for example angled medial-lateral, lateral-medial, anterior-posterior, or posterior-anterior (or intersecting the quadrants therebetween). In some aspects, the fibula may also be considered in the determination of the optimal osteotomy angle (e.g., whether a fibular osteotomy is acceptable or desirable for the physician for a given patient).

In some aspects, the step 62 may also include the determination of an optimal osteotomy angle for a fibular osteotomy. For example, if correction is desired that requires a fibular osteotomy, the software program/system may determine an optimal osteotomy point along a long axis of the fibula and a corresponding osteotomy angle intersecting said osteotomy point. For example, in some aspects the optimal osteotomy angle of the fibula may be parallel and/or coplanar to the osteotomy of the tibia.

The process 50 is shown to include a step 64 of performing an osteotomy at the desired point and along the optimal osteotomy angle, according to an exemplary embodiment. Step 64 may be performed by the physician who provided patient input data in step 52. In some aspects, the physician may review the optimal osteotomy angle determined in step 62 and approve the angle prior to performing the procedure (or reject the determination, in which case the software program/system may determine a secondary optimal osteotomy angle, where this process is iterated until it is approved by the physician). In performing the osteotomy to the tibia, the physician may implement standard instruments (e.g., saws, powered drivers/handsets, etc.) commonly used in orthopedic procedures. In some aspects, step 64 may also include performing a fibular osteotomy.

The process 50 is shown to include a step 66 of manipulating the anatomy to achieve the desired correction, according to an exemplary embodiment. Step 66, which may be performed by the physician intraoperatively, may include rotating a distal portion of the tibia (and anatomy distal of the osteotomy site) according to the desired correction determined previously in the process 50 (and/or the process 10). For example, the physician may rotate the distal portion of the tibia 5 degrees medially, where the desired rotational correction was 5 degrees in the medial direction.

The process 50 is shown to include a step 68 of applying fixation across the osteotomy site, according to an exemplary embodiment. In some aspects, the physician may be provided with one or more fixation options (e.g., plates, intramedullary nails, etc.) and appropriate accessories (e.g., screws and/or other fasteners) configured to fix the tibia (and the fibula if a fibular osteotomy has been performed) in the desired, corrected position. In some aspects, different fixation options may be provided for different angular osteotomies. For example, a kit may include longer plates for spanning osteotomies performed at steeper angles than plates for spanning osteotomies performed at flatter angles.

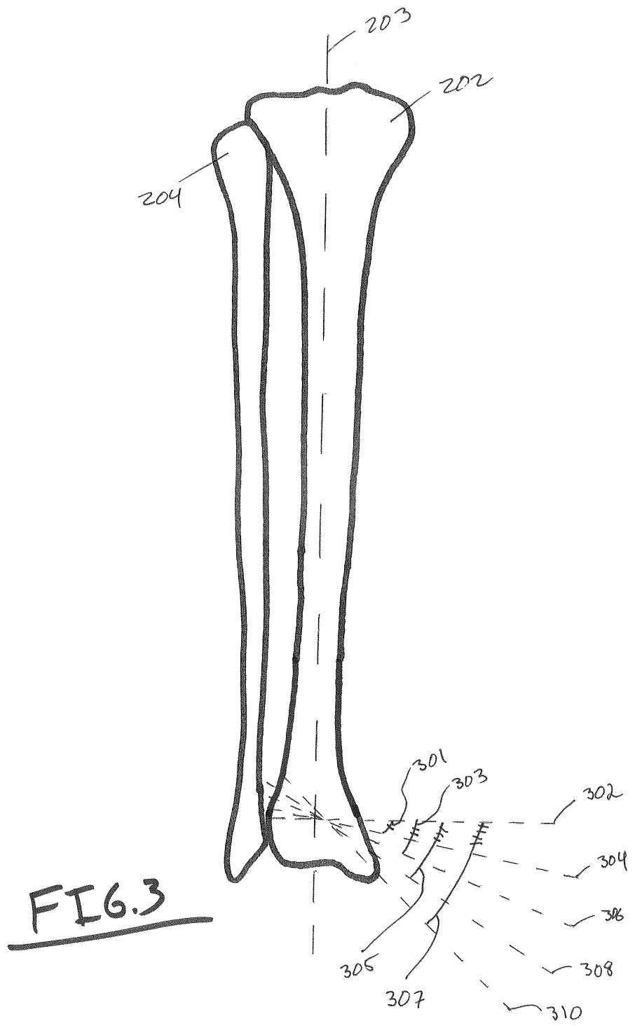

Referring now to FIGS. 3-13, schematic diagrams showing a lower leg of a patient, according to an exemplary embodiment. In some aspects, one or more anatomic landmarks, points, or other features may be identified on a tibia 202 and/or fibula 204, for example a longitudinal axis 203 (axis 203) of the tibia 202. The schematic diagrams of FIGS. 3-13 may be representative of the analysis performed by the software program/system in performing at least step 62 of the process 50 in which an optimal osteotomy angle is determined to correct a deformity of the tibia. Further, it should be understood that the orientation of various osteotomy angles and planes shown and referenced in FIGS. 3-13 are exemplary and not necessarily indicative of orientation. For example, osteotomies of the distal tibia may be performed from a distal-medial point on the tibia 202 with the cut extending upward toward a proximal-lateral point on the tibia 202. In some aspects, the identification of an optimal osteotomy angle and plane may include the software program/system analyzing various factors and outcomes, including those described herein, for start and end points of the osteotomy around at least a portion of the outer perimeter of the tibia 202.

As mentioned with reference to step 62, in determining an optimal osteotomy angle the software program/system may analyze one or more potential osteotomy angles of the tibia 202, with each of said osteotomy angles include at least an axis and/or a plane which intersects the osteotomy point (which is a point at a height determined along the axis 203 of the tibia 202. The schematic diagram of FIG. 3 shows various osteotomy angles which may be considered by the software program/system in determining an optimal osteotomy angle.

In order to determine the optimal osteotomy angle, the software program/system may consider one or more possible osteotomy angles as well as various corresponding measurements, positional outcomes, and other factors. For example, the software program/system may consider total surface area and/or volume of any resulting overhang and/or underhang resulting from performing an osteotomy of the tibia 202 at a particular angle relative to the osteotomy point and subsequently performing a necessary corrective rotation of the distal portion of the tibia 202 (and all anatomy distal of the distal portion of the tibia 202) relative to the proximal portion of the tibia 202. The schematic diagrams of FIGS. 3-13 show an exemplary process (at least a portion of which may be included in one or both of the processes 10, 50 and steps thereof, for example step 62) of analyzing one or more possible osteotomy angles for a single osteotomy point and the subsequent overhang/underhang volume/surface area resulting from various rotations after such an osteotomy is performed.

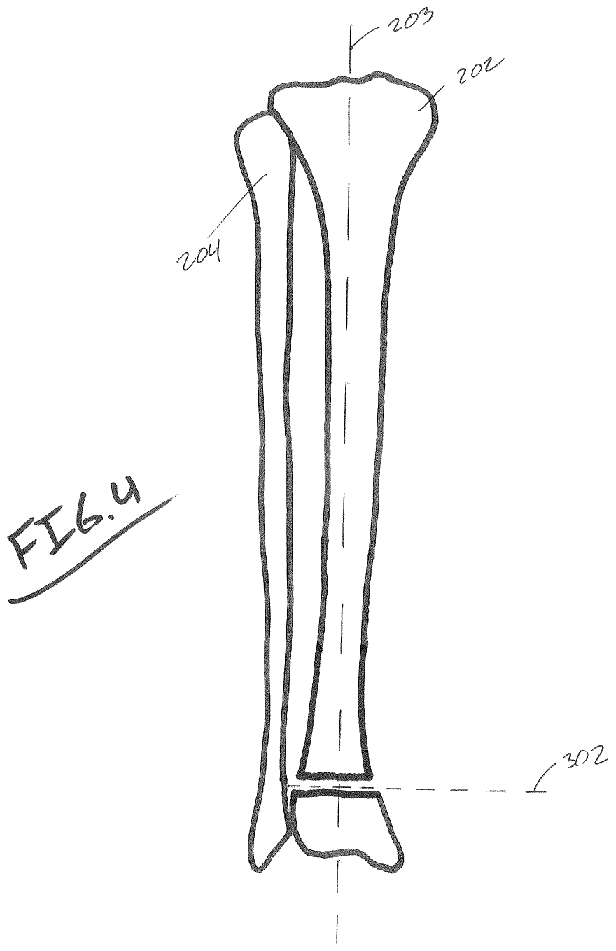

Referring to FIG. 3, a first osteotomy plane 302 (“first plane 302”) is shown relative to the tibia 202 of the patient, according to an exemplary embodiment. The first plane 302 is shown to be substantially orthogonal relative to the axis 203 of the tibia 202. Accordingly, the abutting surfaces of the resultant proximal and distal segments of the tibia 202 created by performing an osteotomy along the first plane 302 are shown in FIG. 4 to be substantially flat relative to one another (e.g., the planes of the abutting surfaces are substantially perpendicular to the axis 203, just as the first plane 302 is).

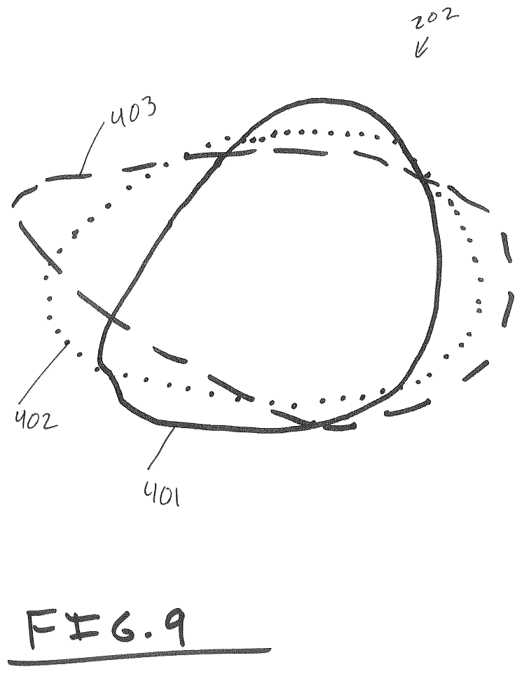

Referring to FIG. 9, an exemplary footprint 401 of the tibia 202 is shown, where the footprint 401 represents the profile (e.g., perimeter of a cross-section) of the proximal portion of the tibia 202 created by performing an osteotomy along the first plane 302. The footprint 401 is shown to be adjacent to a first projection 402, which shows the footprint of the distal portion of the tibia 202 (after performing an osteotomy along the first plane 302) after performing a minor rotational correction. Similarly, the footprint 401 is also shown to be adjacent to a second projection 403, which shows the footprint of the distal portion of the tibia 202 (after performing an osteotomy along the first plane 302) after performing a major rotational correction. The first and second projections 402, 403 are indicative of the change in lateral dimension (e.g., the creation of a prominence at a point which previously did not include a prominence, and/or the repositioning of a prominence about the axis 203 of the tibia 202) of at least a portion of the tibia 202 after an osteotomy is performed along the first plane 302 and the distal portion of the tibia 202 has been repositioned by performing a rotational correction.

In some aspects, the software program/system may analyze the surface area and/or volume of any overhang/underhang resulting from the minor and major rotational corrections shown with respect to the first and second projects 402, 403, respectively. This analysis may be configured to identify an osteotomy plane, through the given osteotomy point, which minimized the surface area and/or volume of any overhang/underhang for a required rotational correction of the distal portion of the tibia 202. Further, the software program/system may also be configured to minimize any prominence on the distal portion of the tibia 202 that extends beyond the footprint of the proximal portion of the tibia 202 so as to minimize any effect on adjacent soft tissue structures. For example, the software program/system may analyze multiple potential osteotomy planes through the osteotomy point and, based on minimizing any resultant footprint of the distal portion of the tibia 202 beyond that of the proximal portion of the tibia 202, identify an optimal osteotomy plane. This analysis may include calculating the total surface area, volume, or change in radial difference from the axis 203/vertex of the footprint of the proximal portion of the tibia 202 to the perimeter of the footprint of the distal portion of the tibia 202). Further, this analysis may also consider the position of soft tissue structures relative to any repositioned projections or prominences created by the rotational correction of the distal portion of the tibia 202 relative to the proximal portion of the tibia 202.

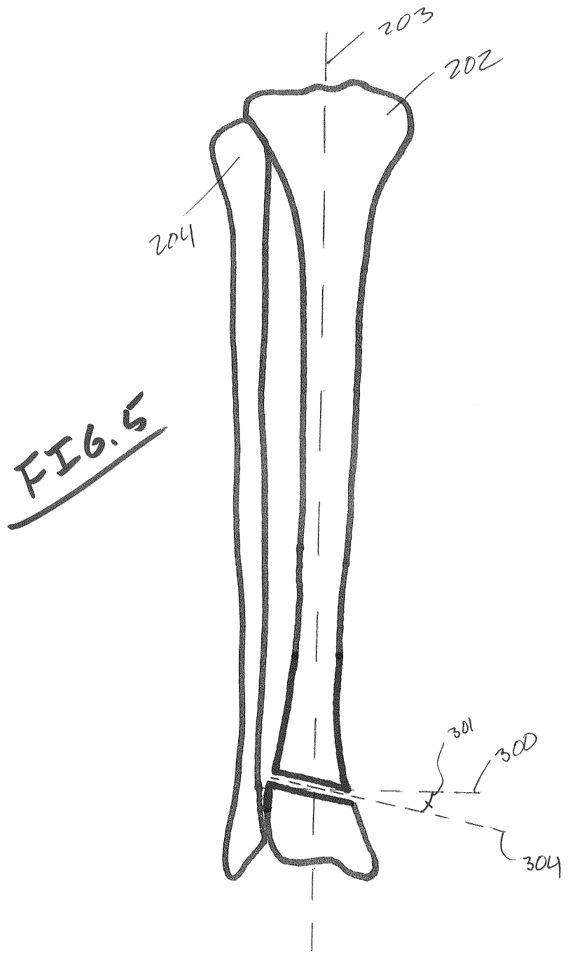

With continued reference to FIG. 3, a second osteotomy plane 304 (“second plane 304”) performed at an angle 301 relative to a horizontal plane 300 (shown in FIG. 5) (where the horizontal plane 300 is parallel to/coplanar with the first plane 302) is shown relative to the tibia 202 of the patient, according to an exemplary embodiment. The second plane 304 is shown to form an approximately 10-degree angle relative to the horizontal plane 300 (e.g., an approximately 80- or 110-degree angle relative to the axis 203 of the tibia 202). Accordingly, the abutting surfaces of the resultant proximal and distal segments of the tibia 202 created by performing an osteotomy along the second plane 304 are shown in FIG. 5 to be substantially oblique and parallel one another (e.g., the planes of the abutting surfaces are oblique relative to the axis 203, just as the second plane 304 is).

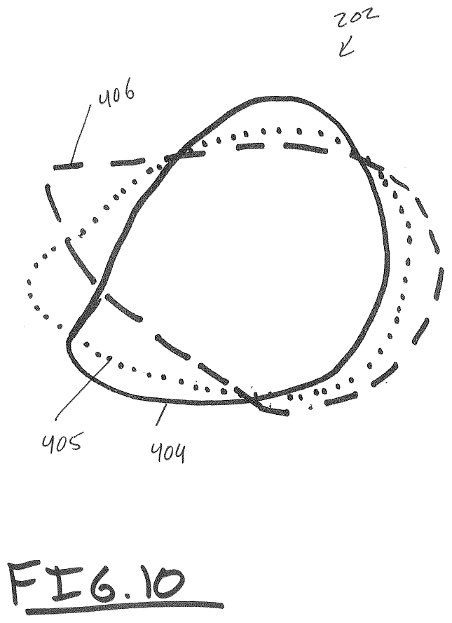

Referring to FIG. 10, an exemplary footprint 404 of the tibia 202 is shown, where the footprint 404 represents the profile (e.g., perimeter of a cross-section) of the proximal portion of the tibia 202 created by performing an osteotomy along the second plane 304. The footprint 404 is shown adjacent to a first projection 405, which shows the footprint of the distal portion of the tibia 202 (after performing an osteotomy along the second plane 304) after performing a minor rotational correction. Similarly, the footprint 404 is also shown adjacent to a second projection 406, which shows the footprint of the distal portion of the tibia 202 (after performing an osteotomy along the second plane 304) after performing a major rotational correction. The first and second projections 405, 406 are indicative of the change in lateral dimension (e.g., the creation of a prominence at a point which previously did not include a prominence, and/or the repositioning of a prominence about the axis 203 of the tibia 202) of at least a portion of the tibia 202 after an osteotomy is performed along the second plane 304 and the distal portion of the tibia 202 has been repositioned by performing a rotational correction. As shown, the overhang/underhang surface area and the radial distance of various points on the perimeter (relative to the axis 203) of the first and second projections 405, 406 are lesser than that of the first and second projections 402, 403 of FIG. 4 when approximately the same rotational corrections are performed. Accordingly, the software program/system may identify the second plane 304 as optimal relative to the first plane 302.

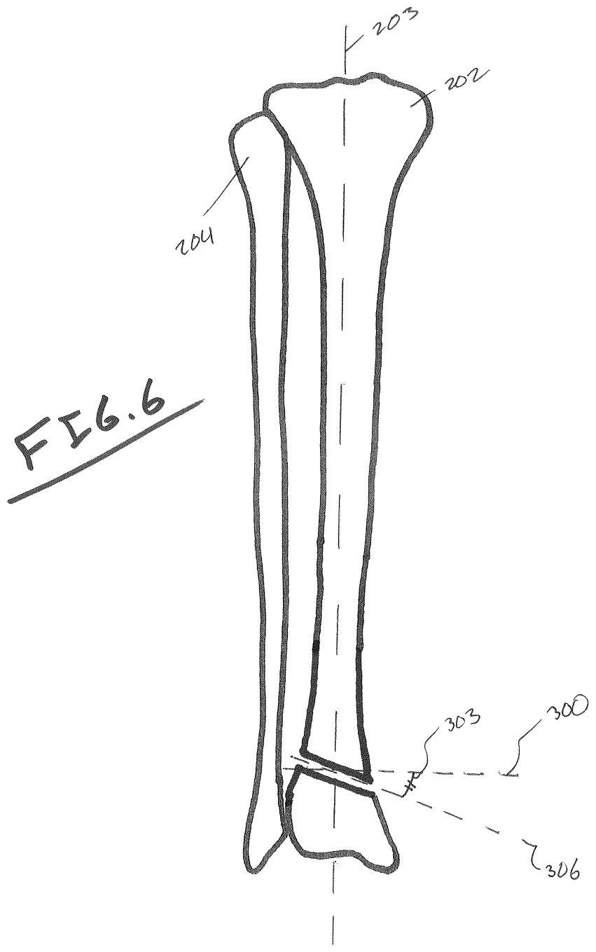

With continued reference to FIG. 3, a third osteotomy plane 306 (“third plane 306”) performed at an angle 303 relative to a horizontal 300 (where the horizontal 300 is parallel to/coplanar with the first plane 302) is shown relative to the tibia 202 of a patient, according to an exemplary embodiment. The third plane 306 is shown to form an approximately 20-degree angle relative to the horizontal plane 300 (e.g., an approximately 70- or 120-degree angle relative to the axis 203 of the tibia 202). Accordingly, the abutting surfaces of the resultant proximal and distal segments of the tibia 202 created by performing an osteotomy along the third plane 306 are shown in FIG. 6 to be substantially oblique and parallel one another (e.g., the planes of the abutting surfaces are oblique relative to the axis 203, just as the third plane 306 is).

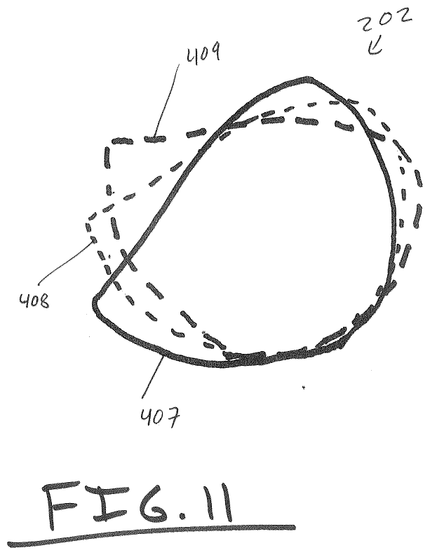

Referring to FIG. 11, an exemplary footprint 407 of the tibia 202 is shown, where the footprint 407 represents the profile (e.g., perimeter of a cross-section) of the proximal portion of the tibia 202 created by performing an osteotomy along the third plane 306. The footprint 407 is shown to be adjacent to a first projection 408, which shows the footprint of the distal portion of the tibia 202 (after performing an osteotomy along the third plane 306) after performing a minor rotational correction. Similarly, the footprint 407 is also shown adjacent to a second projection 409, which shows the footprint of the distal portion of the tibia 202 (after performing an osteotomy along the third plane 306) after performing a major rotational correction. The first and second projections 408, 409 are indicative of the change in lateral dimension (e.g., the creation of a prominence at a point which previously did not include a prominence, and/or the repositioning of a prominence about the axis 203 of the tibia 202) of at least a portion of the tibia 202 after an osteotomy is performed along the third plane 306 and the distal portion of the tibia 202 has been repositioned by performing a rotational correction. As show, the overhang/underhang surface area and the radial distance of the various points on the perimeter (relative to the axis 203) of the first and second projections 408, 409 are lesser than that of the first and second projections 402, 403 of FIG. 4 as well as the first and second projections 405, 406 of FIG. 5 when approximately the same rotational corrections are performed. Accordingly, the software program/system may identify the third plane 306 as optimal relative to the first plane 302 and the second plane 304.

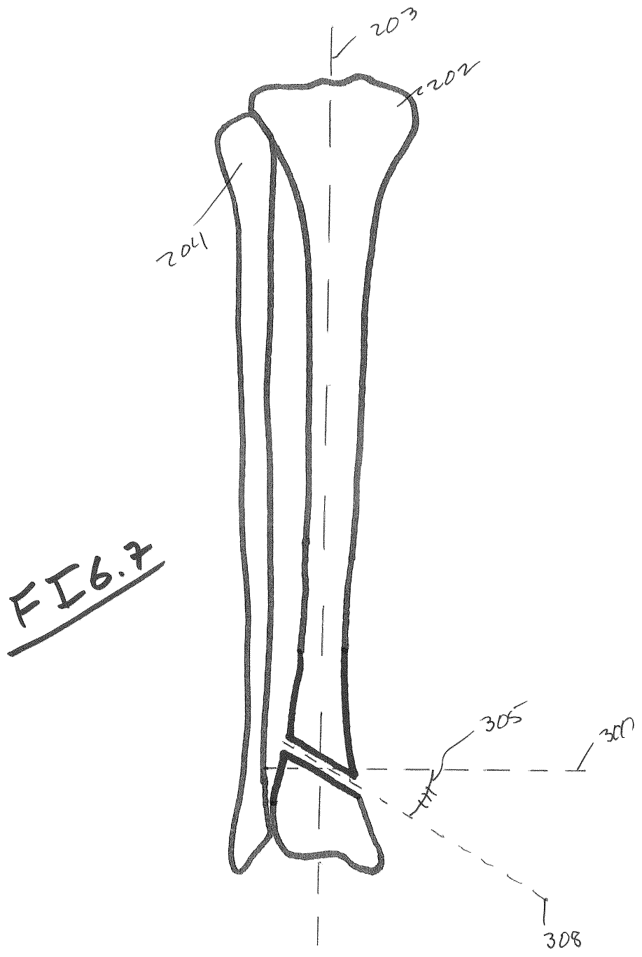

With continued reference to FIG. 3, a fourth osteotomy plane 308 (“fourth plane 308”) performed at an angle 305 relative to the horizontal plane 300 (where the horizontal plane 300 is parallel to/coplanar with the first plane 302) is shown relative to the tibia 202 of the patient, according to an exemplary embodiment. The fourth plane 308 is shown to form an approximately 30-degree angle relative to the horizontal plane 300 (e.g., an approximately 60- or 130-degree angle relative to the axis 203 of the tibia 202). Accordingly, the abutting surfaces of the resultant proximal and distal segments of the tibia 202 created by performing an osteotomy along the fourth plane 308 are shown in FIG. 7 to be substantially oblique and parallel one another (e.g., the planes of the abutting surfaces are oblique relative to the axis 203, just as the fourth plane 308 is).

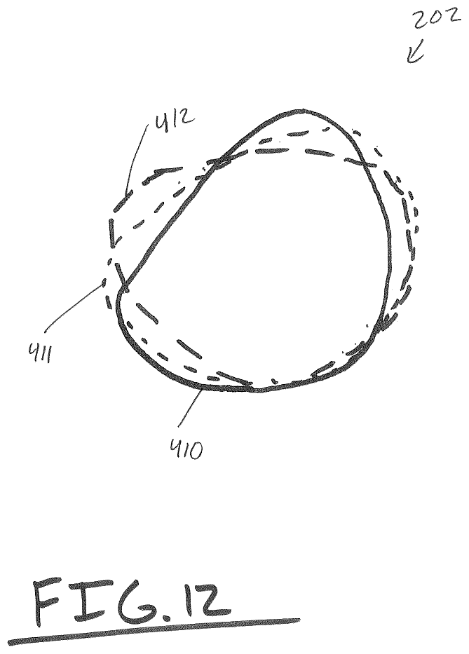

Referring to FIG. 12, an exemplary footprint 410 of the tibia 202 is shown, where the footprint 410 represents the profile (e.g., perimeter of a cross-section) of the proximal portion of the tibia 202 created by performing an osteotomy along the fourth plane 308. The footprint 410 is shown to be adjacent to a first projection 411, which shows the footprint of the distal portion of the tibia 202 (after performing an osteotomy along the fourth plane 308) after performing a minor rotational correction. Similarly, the footprint 410 is also shown to be to be adjacent to a second projection 412, which shows the footprint of the distal portion of the tibia 202 (after performing an osteotomy along the fourth plane 308) after performing a major rotational correction. The first and second projections 411, 412 are indicative of the change in lateral dimension (e.g., the creation of a prominence at a point which previously did not include a prominence, and/or the repositioning of a prominence about the axis 203 of the tibia 202) of at least a portion of the tibia 202 after an osteotomy is performed along the fourth plane 308 and the distal portion of the tibia 202 has been repositioned by performing a rotational correction. As shown, the overhang/underhang surface area and the radial distance of various points on the perimeter (relative to the axis 203) of the first and second projections 411, 412 are lesser than that of the first and second projections 402, 403 of FIG. 4, the first and second projections 405, 406 of FIG. 5, and the first and second projections 408, 409 of FIG. 6 when approximately the same rotational corrections are performed. Accordingly, the software program/system may identify the fourth plane 308 as optimal relative to the first plane 302, the second plane 304, and the third plane 306.

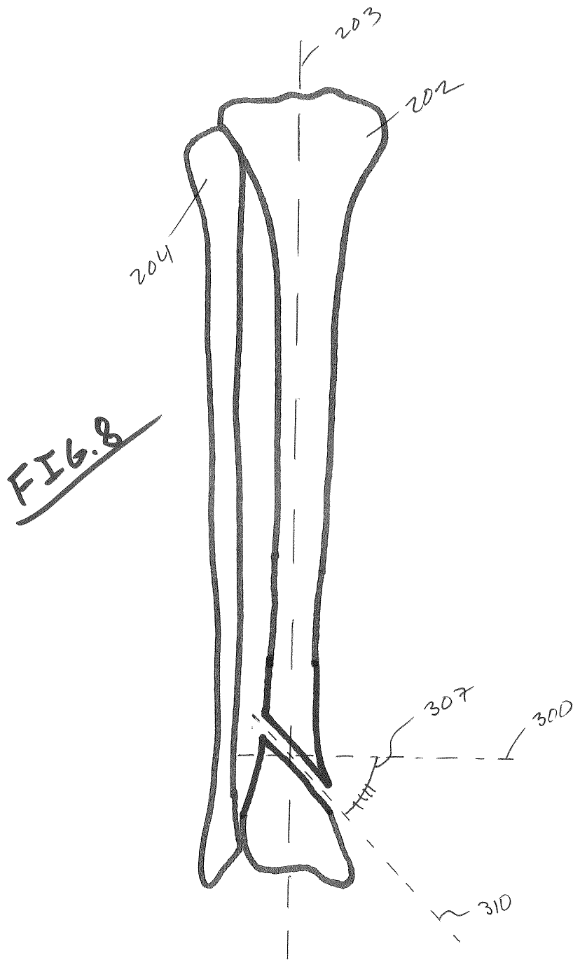

With continued reference to FIG. 3, a fifth osteotomy plane 310 (“fifth plane 310”) performed at an angle 307 relative to a horizontal plane 300 (where the horizontal plane 300 is parallel to/coplanar with the first plane 302) is shown relative to the tibia 202 of the patient, according to an exemplary embodiment. The fifth plane 310 is shown to form an approximately 40-degree angle relative to the horizontal plane 300 (e.g., an approximately 50- or 140-degree angle relative to the axis 203 of the tibia 202). Accordingly, the abutting surfaces of the resultant proximal and distal segments of the tibia 202 created by performing an osteotomy along the fifth plane 310 are shown in FIG. 8 to be substantially oblique and parallel one another (e.g., the planes of the abutting surfaces are oblique relative to the axis 203, just as the fifth plane 310 is).

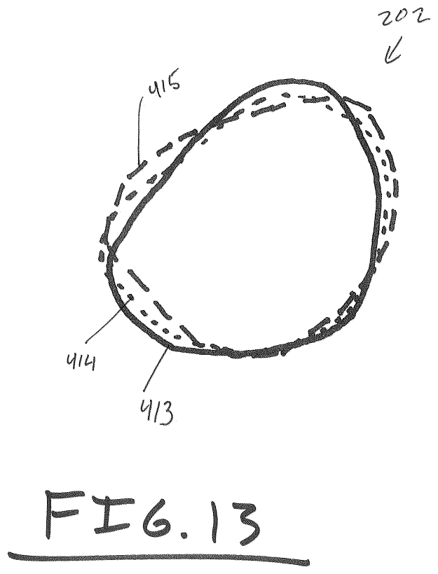

Referring to FIG. 13, an exemplary footprint 413 of the tibia 202 is shown, where the footprint 413 represents the profile (e.g., perimeter of a cross-section) of the proximal portion of the tibia 202 created by performing an osteotomy along the fifth plane 310. The footprint 413 is shown to be adjacent to a first projection 414, which shows the footprint of the distal portion of the tibia 202 (after performing an osteotomy along the fifth plane 310) after performing a minor rotational correction. Similarly, the footprint 413 is also shown to be adjacent to a second projection 415, which shows the footprint of the distal portion of the tibia 202 (after performing an osteotomy along the fifth plane 310) after performing a major rotational correction. The first and second projections 414, 415 are indicative of the change in lateral dimension (e.g., the creation of a prominence at a point which previously did not include a prominence, and/or the repositioning of a prominence about the axis 203 of the tibia 202) of at least a portion of the tibia 202 after an osteotomy is performed along the fifth plane 310 and the distal portion of the tibia 202 has been repositioned by performing a rotational correction. As show, the overhang/underhang surface area and the radial distance of various points on the perimeter (relative to the axis 203) of the first and second projections 414, 415 are lesser than that of the first and second projections 402, 403 of FIG. 4, the first and second projections 405, 406 of FIG. 5, the first and second projections 408, 409 of FIG. 6, and the first and second projections 411, 412 of FIG. 7 when approximately the same rotational corrections are performed. Accordingly, the software program/system may identify the fifth plane 310 as optimal relative to the first plane 302, the second plane 304, the third plane 306, and the fourth plane 308.

Accordingly, in analyzing the first, second, third, fourth, and fifth osteotomy planes 302, 304, 306, 308, and 310, the software program/system may calculate one or more metrics relative to one or more rotational corrections of the distal portion of the tibia 202 relative to the proximal portion of the tibia 202 for each of the osteotomy planes. Further, the software program/system may then determine an optimal osteotomy plane from those analyzed based on minimizing one or more of the calculated metrics relative to the overhang/underhang surface area and/or volume, radial distance of one or more points about the perimeter of the footprint of the distal portion of the tibia 202 relative to the axis 203, location of one or more soft tissue structures relative to the perimeter of the footprint of the distal portion of the tibia 202, as well as other metrics which may also be calculated or considered. It should be understood that the first, second, third, fourth, and fifth osteotomy planes 302, 304, 306, 308, and 310 are exemplary in nature, and the software program/system may simulate more or fewer osteotomy planes for one or more osteotomy points along the axis 203. For example, the software program/system may simulate osteotomy planes at intervals of 0.1 degrees, 1 degree, 2 degrees, 5 degrees, or other intervals. Further, it should be understood that the software program/system may also simulate the high and low points of any angled osteotomy planes about the outer perimeter of the tibia 202 (e.g., where an osteotomy in the second plane 304 may be rotated about the axis 203 such that the high and low points of the second plane 304 may be placed variously about the outer perimeter of the tibia 202).

In some aspects, the software program/system described herein may include one or more modules having AI capabilities. For example, the software program/system may include an optimization module which has been trained to identify an optimal osteotomy angle or plane based on a database of data of previously performed tibial osteotomies. In analyzing various properties/features of the osteotomies (for example, total surface area and/or volume of any overhang/underhang, increases/changes in radial distance of the outer perimeter of bony segments for a given rotational correction, or positional changes of features of the bony anatomy as a result of a given rotational correction, etc.) this optimization module may leverage prior-performed procedures and data thereof, for instance as training data to train AI-based model(s), in order to identify the optimal and/or recommended osteotomy angle and/or plane.

In some aspects, the processes 10 and/or 50 may include the design and creation of patient-specific instrumentation (PSI), for example PSI guides configured to guide a saw blade in performing an osteotomy. For example, after an optimal osteotomy plane has been identified, the software program/system may generate a PSI cut guide configured to interface with at least a portion of the bony anatomy of the patient and guide a physician in manipulating a saw blade within a slot of the guide so as to ensure that the saw cuts the bone along the optimal osteotomy angle. In some aspects, these PSI guides may include one or more surface matching features, for example complimentary geometric features to the surface anatomy of the tibia of the patient so as to facilitate fitting and retention of the PSI guide in the desired position.

Processes (including processes, methods and/or other aspects described herein) may be executed/performed by processor(s) and/or processing circuit(s), for instance those of one or more computers/computer systems, such as those described herein. For instance, code or instructions implementing processes described herein may be part of modules of software/computer program(s) for execution by one or more computer systems.

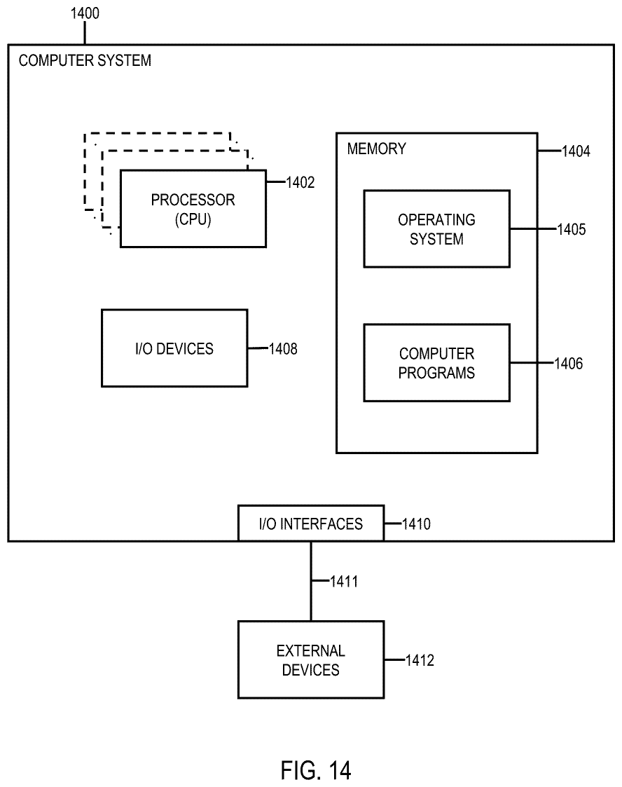

Accordingly, processes described herein, including aspects of those processes, may be performed singly or collectively by one or more computer systems, such as one or more computer systems executing surgical planning software, as an example. FIG. 14 depicts one example of such a computer system and associated devices to incorporate and/or use aspects described herein. A computer system may also be referred to herein as a data processing device/system, computing device/system/node, or simply a computer. The computer system may be based on one or more of various system architectures and/or instruction set architectures, such as those offered by Intel Corporation (Santa Clara, California, USA) or ARM Holdings plc (Cambridge, England, United Kingdom), as examples.

FIG. 14 shows a computer system 1400 in communication with external device(s) 1412. Computer system 1400 includes one or more processor(s) 1402, for instance central processing unit(s) (CPUs). A processor can include functional components used in the execution of instructions, such as functional components to fetch program instructions from locations such as cache or main memory, decode program instructions, and execute program instructions, access memory for instruction execution, and write results of the executed instructions. A processor 1402 can also include register(s) to be used by one or more of the functional components. Computer system 1400 also includes memory 1404, input/output (I/O) devices 1408, and I/O interfaces 1410, which may be coupled to processor(s) 1402 and each other via one or more buses and/or other connections. Bus connections represent one or more of any of several types of bus structures, including a memory bus or memory controller, a peripheral bus, an accelerated graphics port, and a processor or local bus using any of a variety of bus architectures. By way of example, and not limitation, such architectures include the Industry Standard Architecture (ISA), the Micro Channel Architecture (MCA), the Enhanced ISA (EISA), the Video Electronics Standards Association (VESA) local bus, and the Peripheral Component Interconnect (PCI).

Memory 1404 can be or include main or system memory (e.g. Random Access Memory) used in the execution of program instructions, storage device(s) such as hard drive(s), flash media, or optical media as examples, and/or cache memory, as examples. Memory 1404 can include, for instance, a cache, such as a shared cache, which may be coupled to local caches (examples include L1 cache, L2 cache, etc.) of processor(s) 1402. Additionally, memory 1404 may be or include at least one computer program product having a set (e.g., at least one) of program modules, instructions, code or the like that is/are configured to carry out functions of embodiments described herein when executed by one or more processors.

Memory 1404 can store an operating system 1405 and other computer programs 1406, such as one or more computer programs/applications that execute to perform aspects described herein. Specifically, programs/applications can include computer readable program instructions that may be configured to carry out functions of embodiments of aspects described herein.

Examples of I/O devices 1408 include but are not limited to microphones, speakers, Global Positioning System (GPS) devices, cameras, lights, accelerometers, gyroscopes, magnetometers, sensor devices configured to sense light, proximity, heart rate, body and/or ambient temperature, blood pressure, and/or skin resistance, and activity monitors. An I/O device may be incorporated into the computer system as shown, though in some embodiments an I/O device may be regarded as an external device (1412) coupled to the computer system through one or more I/O interfaces 1410.

Computer system 1400 may communicate with one or more external devices 1412 via one or more I/O interfaces 1410. Example external devices include a keyboard, a pointing device, a display, and/or any other devices that enable a user to interact with computer system 1400. Other example external devices include any device that enables computer system 1400 to communicate with one or more other computing systems or peripheral devices such as a printer. A network interface/adapter is an example I/O interface that enables computer system 1400 to communicate with one or more networks, such as a local area network (LAN), a general wide area network (WAN), and/or a public network (e.g., the Internet), providing communication with other computing devices or systems, storage devices, or the like. Ethernet-based (such as Wi-Fi) interfaces and Bluetooth® adapters are just examples of the currently available types of network adapters used in computer systems (BLUETOOTH is a registered trademark of Bluetooth SIG, Inc., Kirkland, Washington, U.S.A.).

The communication between I/O interfaces 1410 and external devices 1412 can occur across wired and/or wireless communications link(s) 1411, such as Ethernet-based wired or wireless connections. Example wireless connections include cellular, Wi-Fi, Bluetooth®, proximity-based, near-field, or other types of wireless connections. More generally, communications link(s) 1411 may be any appropriate wireless and/or wired communication link(s) for communicating data.

Particular external device(s) 1412 may include one or more data storage devices, which may store one or more programs, one or more computer readable program instructions, and/or data, etc. Computer system 1400 may include and/or be coupled to and in communication with (e.g. as an external device of the computer system) removable/non-removable, volatile/non-volatile computer system storage media. For example, it may include and/or be coupled to a non-removable, non-volatile magnetic media (typically called a “hard drive”), a magnetic disk drive for reading from and writing to a removable, non-volatile magnetic disk (e.g., a “floppy disk”), and/or an optical disk drive for reading from or writing to a removable, non-volatile optical disk, such as a CD-ROM, DVD-ROM or other optical media.

Computer system 1400 may be operational with numerous other general purpose or special purpose computing system environments or configurations. Computer system 1400 may take any of various forms, well-known examples of which include, but are not limited to, personal computer (PC) system(s), server computer system(s), such as messaging server(s), thin client(s), thick client(s), workstation(s), laptop(s), handheld device(s), mobile device(s)/computer(s) such as smartphone(s), tablet(s), and wearable device(s), multiprocessor system(s), microprocessor-based system(s), telephony device(s), network appliance(s) (such as edge appliance(s)), virtualization device(s), storage controller(s), set top box(es), programmable consumer electronic(s), network PC(s), minicomputer system(s), mainframe computer system(s), and distributed cloud computing environment(s) that include any of the above systems or devices, and the like.

Aspects of the present invention may be a system, a method, and/or a computer program product, any of which may be configured to perform or facilitate aspects described herein.

In some embodiments, aspects of the present invention may take the form of a computer program product, which may be embodied as computer readable medium(s). A computer readable medium may be a tangible storage device/medium having computer readable program code/instructions stored thereon. Example computer readable medium(s) include, but are not limited to, electronic, magnetic, optical, or semiconductor storage devices or systems, or any combination of the foregoing. Example embodiments of a computer readable medium include a hard drive or other mass-storage device, an electrical connection having wires, random access memory (RAM), read-only memory (ROM), erasable-programmable read-only memory such as EPROM or flash memory, an optical fiber, a portable computer disk/diskette, such as a compact disc read-only memory (CD-ROM) or Digital Versatile Disc (DVD), an optical storage device, a magnetic storage device, or any combination of the foregoing. The computer readable medium may be readable by a processor, processing unit, or the like, to obtain data (e.g. instructions) from the medium for execution. In a particular example, a computer program product is or includes one or more computer readable media that includes/stores computer readable program code to provide and facilitate one or more aspects described herein.

As noted, program instruction contained or stored in/on a computer readable medium can be obtained and executed by any of various suitable components such as a processor of a computer system to cause the computer system to behave and function in a particular manner. Such program instructions for carrying out operations to perform, achieve, or facilitate aspects described herein may be written in, or compiled from code written in, any desired programming language. In some embodiments, such programming language includes object-oriented and/or procedural programming languages such as C, C++, C#, Java, etc.

Program code can include one or more program instructions obtained for execution by one or more processors. Computer program instructions may be provided to one or more processors of, e.g., one or more computer systems, to produce a machine, such that the program instructions, when executed by the one or more processors, perform, achieve, or facilitate aspects of the present invention, such as actions or functions described in flowcharts and/or block diagrams described herein. Thus, each block, or combinations of blocks, of the flowchart illustrations and/or block diagrams depicted and described herein can be implemented, in some embodiments, by computer program instructions.

Although various embodiments are described above, these are only examples.

The terminology used herein for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

What is claimed is:

1. A process for identifying a deformity, comprising:
receiving patient input data showing patient anatomy;
segmenting the patient input data;
generating a three-dimensional model of the patient anatomy;
collecting one or more measurements from the three-dimensional model of the patient anatomy; and
determining if a deformity is present in the patient anatomy.

2. The process of claim 1, wherein the patient anatomy includes a tibia of a patient.

3. The process of claim 2, further comprising:
identifying a proximal portion of the tibia in the three-dimensional model; and
identifying a distal portion of the tibia in the three-dimensional model.

4. The process of claim 3, further comprising:
identifying one or more anatomic or geometric planes relative to the tibia in the three-dimensional model.

5. The process of claim 4, further comprising:
identifying one or more points on the proximal portion of the tibia in the three-dimensional model; and
identifying one or more points on the distal portion of the tibia in the three-dimensional model.

6. The process of claim 5, wherein the collecting one or more measurements from the three-dimensional model of the patient anatomy further comprises:
determining the position of the one or more points on the proximal portion of the tibia relative to the one or more anatomic landmarks or geometric planes in the three-dimensional model; and
determining the position of the one or more points on the distal portion of the tibia relative to the one or more anatomic landmarks or geometric planes in the three-dimensional model.

7. The process of claim 6, further comprising:
calculating a difference between the determined position of the one or more points on the proximal portion of the tibia relative to the one or more anatomic landmarks or geometric planes in the three-dimensional model and a normal position of the one or more points on the proximal portion of the tibia relative to the one or more anatomic landmarks or geometric planes.

8. The process of claim 7, further comprising:
calculating a difference between the determined position of the one or more points on the distal portion of the tibia relative to the one or more anatomic landmarks or geometric planes in the three-dimensional model and a normal position of the one or more points on the distal portion of the tibia relative to the one or more anatomic landmarks or geometric planes.

9. The process of claim 8, wherein the determining if a deformity is present in the patient anatomy further comprises:
determining, based on the calculated difference between the determined position of the one or more points on the proximal portion of the tibia relative to the one or more anatomic landmarks or geometric planes in the three-dimensional model and the normal position of the one or more points on the proximal portion of the tibia relative to the one or more anatomic landmarks or geometric planes, if a deformity is present in the proximal portion of the tibia.

10. The process of claim 9, wherein the determining if a deformity is present in the patient anatomy further comprises:
determining, based on the calculated difference between the determined position of the one or more points on the distal portion of the tibia relative to the one or more anatomic landmarks or geometric planes in the three-dimensional model and the normal position of the one or more points on the distal portion of the tibia relative to the one or more anatomic landmarks or geometric planes, if a deformity is present in the proximal portion of the tibia.

11. The process of claim 10, further comprising:
classifying a deformity identified in the proximal portion of the tibia.

12. The process of claim 10, further comprising:
classifying a deformity identified in the distal portion of the tibia.

13. The process of claim 12, further comprising:
recommending, based on the deformity identified in the distal portion of the tibia, one or more corrective procedures to be performed to the tibia.

14. A process for determining an optimal osteotomy angle, comprising:
receiving patient input data showing patient anatomy;
segmenting the patient input data;
generating a three-dimensional model of the patient anatomy;
identifying one or more anatomic structures in the three-dimensional model;
identifying one or more anatomic landmarks on the one or more anatomic structures in the three-dimensional model;
determining a desired correction to be achieved from an osteotomy; and
determining an optimal osteotomy angle.

15. The process of claim 14, wherein the desired correction comprises a desired rotational correction of a distal portion of a tibia relative to a proximal portion of the tibia.

16. The process of claim 15, further comprising:
determining an optimal osteotomy point, wherein the optimal osteotomy point is positioned along the long axis of the tibia.

17. The process of claim 16, wherein determining an optimal osteotomy angle comprises:
determining, based on the osteotomy point, total surface area of overhang and underhang resulting from rotation of the distal portion of the tibia relative to the proximal portion of the tibia for an osteotomy performed at a first osteotomy angle across the optimal osteotomy point.

18. The process of claim 17, wherein determining an optimal osteotomy angle comprises: determining, based on the osteotomy point, total surface area of overhang and underhang resulting from rotation of the distal portion of the tibia relative to the proximal portion of the tibia for an osteotomy performed at a second osteotomy angle across the optimal osteotomy point

19. The process of claim 19, wherein determining the optimal osteotomy angle further comprises:
determining, based on the total surface area of overhang and underhang for osteotomies performed at the first and second osteotomy angles, an optimal osteotomy angle,
wherein the optimal osteotomy angle is the angle of the first and second osteotomy angles with the lease total surface area of overhang and underhang.

20. A process for identifying a deformity, comprising:
receiving patient input data showing patient anatomy;
segmenting the patient input data;
generating a three-dimensional model of the patient anatomy;
identifying one or more points on the patient anatomy;
collecting one or more measurements incorporating the one or more points on the patient anatomy;
determining, based on the one or more measurements, if a deformity is present in the patient anatomy; and
recommending one or more actions to correct the deformity of the patient anatomy.
